(19) United States
(12) Patent Application Publication
(10) Pub. No.: US 2019/0376144 A1
(43) Pub. Date: Dec. 12, 2019

(54) DIAGNOSIS, PROGNOSIS AND TREATMENT OF ACUTE MYELOID LEUKEMIA

(71) Applicant: ONCONOSTIC TECHNOLOGIES, INC., Champaign, IL (US)

(72) Inventor: Partha S. RAY, Urbana, IL (US)

(21) Appl. No.: 16/425,848

(22) Filed: May 29, 2019

Related U.S. Application Data
(63) Continuation of application No. PCT/US2017/063785, filed on Nov. 29, 2017.
(60) Provisional application No. 62/428,439, filed on Nov. 30, 2016.

(51) Int. Cl. C12Q 1/6886

(52) U.S. Cl. CPC C12Q 1/6886; C12Q 2600/112; C12Q 2600/158; C12Q 2600/106

(57) ABSTRACT

Disclosed herein is a method of screening AML patients who are unlikely to respond to or are not responsive to induction chemotherapy. The method includes detecting the expression level of FOXC1 in a sample obtained from the AML patient, and an elevated expression level indicates that the AML patient is unlikely to respond to or is not responsive to induction chemotherapy. Also disclosed herein is a method of treating AML patients who are unlikely to respond to or are not responsive to induction chemotherapy. The method includes detecting the expression level of FOXC1 in a sample obtained from the AML patient, and administering a therapeutically effective amount of one or more alternative therapies to the AML patient who has an elevated level of FOXC1 expression. The alternative therapy includes, for example, stem cell transplantation, radiotherapy, or a targeted therapy.

### CROSS-REFERENCE TO RELATED APPLICATIONS

This application is a continuation of International Patent Application No. PCT/US2017/063785, filed Nov. 29, 2017, which claims the benefit of U.S. Provisional Patent Application No. 62/428,439, filed Nov. 30, 2016, both of which are incorporated herein by reference in their entirety.

### BACKGROUND

Acute myeloid leukemia (AML) is a fast-growing cancer of the blood and bone marrow and is the most common type of acute leukemia. Immediate treatment is crucial after diagnosis of AML because cancer cells can spread rapidly to various body parts through the bloodstream. The treatment options for AML include, for example, chemotherapy, and/or stem cell transplantation. In general, the course of therapy includes several months of chemotherapy to induce remission, followed by post-remission chemotherapy and/or autologous or allogeneic stem cell transplantation. However, not all AML patients respond to induction chemotherapy. If all patients are subjected to the same course of treatment, the treatment for patients not responding to chemotherapy is delayed even more because it takes several months to go through chemotherapy and additional several months or even longer to find matching donor(s) and to produce a sufficient amount of autologous stem cells. There is a need to accurately and quickly determine at an early stage the AML patients who are unlikely to respond to chemotherapy, thereby to effectively treat these patients with an appropriate, alternative therapy.

### BRIEF DESCRIPTION OF THE DRAWINGS

FIG. 1 illustrates FOXC1 expression quartiles in overall survival (OS) of AML patients subjected to induction chemotherapy.

FIG. 2 illustrates FOXC1 expression quartiles in event-free survival (EFS) of AML patients subjected to induction chemotherapy.

FIG. 3 illustrates mean FOXC1 mRNA expression level in AML patients subjected to induction chemotherapy.

### DETAILED DESCRIPTION

The following description provides specific details for a thorough understanding of, and enabling description for, embodiments of the disclosure. However, one skilled in the art will understand that the disclosure may be practiced without these details. In other instances, well-known structures and functions have not been shown or described in detail to avoid unnecessarily obscuring the description of the embodiments of the disclosure.

The mesenchymal transcription factor Forkhead Box C1 (FOXC1), derepressed in hematopoietic stem progenitor cells (HSPCs) in the setting of acute myeloid leukemia (AML), but not in normal HSPCs, was recently demonstrated to play an important role in AML disease onset and progression by blocking myeloid lineage differentiation and enhancing clonogenic potential.

As provided herein are methods for screening a population of AML patients to determine prognosis of AML, disease relapse, and/or lack of response to induction chemotherapy in AML patients. Thus, this disclosure relates to performing a simple assay of FOXC1 mRNA expression level, thereby to determine prognosis of AML, disease relapse, and/or lack of response to induction chemotherapy in AML patients. FOXC1 expression in AML patients is an independent prognostic predictor of decreased overall survival (OS) and event-free survival (EFS) and is significantly associated with disease relapse and/or refractoriness to induction chemotherapy. A risk of relapse/non-response score that incorporates FOXC1 expression status can be used to identify patients at the time of initial diagnosis who are likely to fail induction chemotherapy. In some embodiments, the AML patients are adults who are about 60 years old or younger.

In one aspect, this disclosure relates to a method of screening AML patients who are unlikely to respond to or who are non-responsive to induction chemotherapy. The method includes detecting the expression level of FOXC1 from a sample obtained from an AML patient, wherein an elevated expression level of FOXC1 indicates that the patient is unlikely to respond to or is non-responsive to induction chemotherapy. In some embodiments, the sample is a tissue sample or a body fluid sample. In some embodiments, the sample is a bone marrow sample. In some embodiments, the sample is a blood sample. In some embodiments, the AML patient has been subjected to induction chemotherapy. In other embodiments, the AML patient has been diagnosed with AML but has not been subjected to induction chemotherapy.

In another aspect, this disclosure relates to a method of treating an AML patient who is unlikely to respond to or who is non-responsive to induction chemotherapy. For patients who are non-responders to chemotherapy, one or more alternative therapies other than chemotherapy, such as stem cell transplantation, radiotherapy, a targeted therapy, etc., can be administered to the non-responders. In some embodiments, the method includes detecting the expression level of FOXC1 from a sample obtained from an AML patient, and administering a therapeutically effective amount of stem cells to the AML patient who has an elevated expression level of FOXC1. In some embodiments, the sample is a tissue sample or a body fluid sample. In some embodiments, the sample is a bone marrow sample. In some embodiments, the sample is a blood sample. In some embodiments, the AML patient has been subjected to induction chemotherapy. In other embodiments, the AML patient has been diagnosed with AML but has not been subjected to induction chemotherapy. In some embodiments, the stem cells are autologous stem cells. In other embodiments, the stem cells are allogeneic stem cells.

In a related aspect, this disclosure relates to a method of screening and/or treating an AML patient who is likely to respond to or who is responsive to induction chemotherapy. The method includes detecting the expression level of FOXC1 from a sample obtained from an AML patient, and/or administering a therapeutically effective amount of chemotherapy to the AML patient who does not have an elevated expression level of FOXC1. In some embodiments, the sample is a tissue sample or a body fluid sample. In some embodiments, the sample is a bone marrow sample. In some embodiments, the sample is a blood sample. In some embodiments, the AML patient has been subjected to induction chemotherapy. In other embodiments, the AML patient has been diagnosed with AML but has not been subjected to induction chemotherapy.

As provided herein, methods used to determine the expression level of FOXC1 may include any suitable method, including but not limited to, immunohistochemistry (IHC) or other immunoassay, PCR, RT-PCR, qRT-PCR or any other PCR-based method. In some embodiments, the expression level of FOXC1 is determined by IHC. In some embodiments, the expression level of FOXC1 is determined by qRT-PCR.

“Treating” or “treatment” of a condition may refer to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof. Treatment may also mean a prophylactic or preventative treatment of a condition or a prophylactic or preventative treatment of the relapse of a condition.

The phrase “therapeutically effective amount” as used herein refers to an amount of an agent, population of cells, or composition that produces a desired therapeutic effect. For example, a therapeutically effective amount of donor or autologous stem cells may refer to that amount that generates a therapeutic effect in a recipient such that the recipient shows an improvement in his/her cancer condition. The precise therapeutically effective amount is an amount of the agent, population of cells, or composition that will yield the most effective results in terms of efficacy in a given patient. This amount will vary depending upon a variety of factors, including but not limited to the characteristics of the therapeutic agent, population of cells, or composition (including activity, pharmacokinetics, pharmacodynamics, and bioavailability), the physiological condition of the patient (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, namely by monitoring a patient's response to administration of an agent, population of cells, or composition and adjusting the dosage accordingly. For additional guidance, see Remington: The Science and Practice of Pharmacy (Gennaro ed. 20th edition, Williams & Wilkins PA, USA) (2000).

The following example(s) are intended to illustrate various embodiments of the invention. As such, the specific embodiments discussed are not to be construed as limitations on the scope of the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entireties, as if fully set forth herein.

### EXAMPLE(S)

### Example 1

### Detection of FOXC1 mRNA Levels in AML Patients

Method Expression of FOXC1 mRNA in mixed karyotype AML bone marrow samples was examined using publicly available microarray datasets (n=521, n=244). The clinical significance of FOXC1 gene expression as a prognostic biomarker was evaluated using censored overall survival (OS) and event-free survival (EFS) data. Degree of statistical significance in the univariate and multivariate analyses performed was assessed using log-rank test and Cox regression model, respectively.

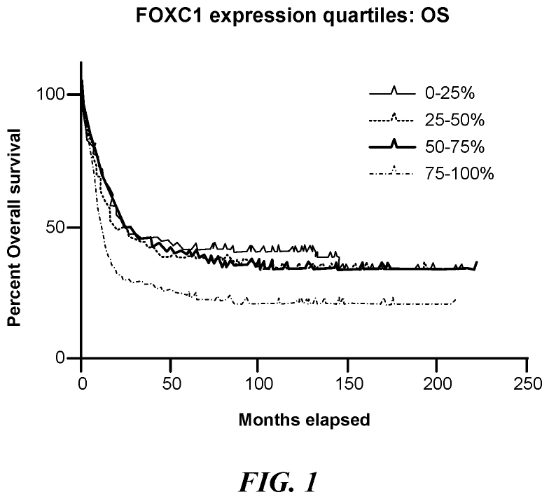

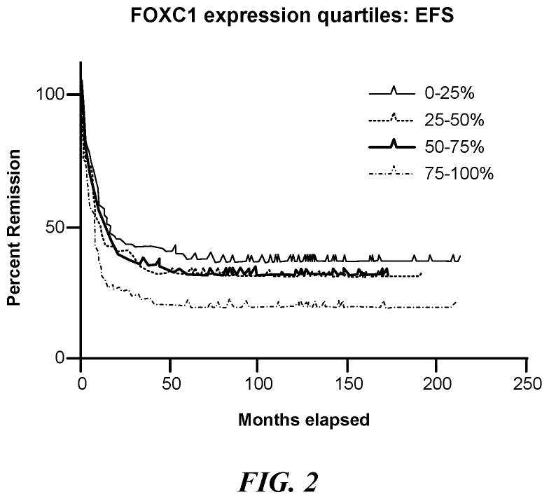

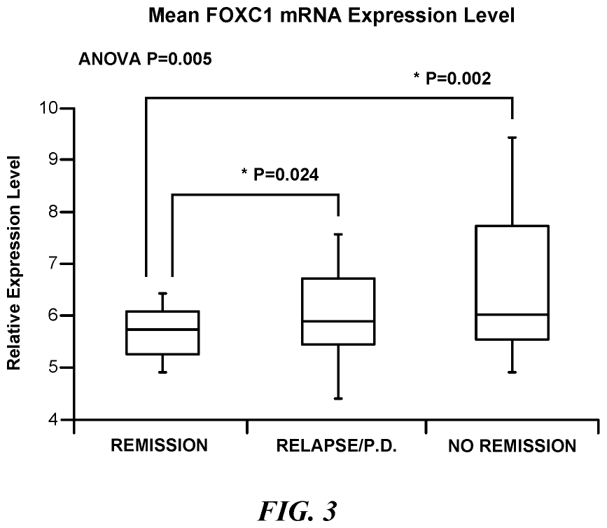

Results As shown in FIGS. 1-3, FOXC1 mRNA expression was a significant predictor of OS on univariate (hazard ratio [HR] 1.592 95% confidence interval [CI] 1.263-2.007, P=0.0001) and multivariate (HR 1.755 95% CI 1.355-2.273, P<0.0001) analyses. This effect on OS could be attributed to disease relapse and/or lack of response to induction chemotherapy as FOXC1 mRNA expression also proved to be a significant predictor of EFS on univariate analysis (hazard ration [HR] 1.539 95% confidence interval [CI] 1.208-1.961, P=0.0002) and multivariate analysis (HR 1.678 95% CI 1.280-2.201, P=0.0001), independent of age, FLT3 ITD status, NPM1 status or cytogenetic risk status. Compared to patients who experienced disease remission, FOXC1 expression was significantly elevated in patients experiencing disease relapse following induction chemotherapy (P<0.02), and in patients who were non-responders to induction chemotherapy (P<0.002).

1. A method of screening for an AML patient who is unlikely to respond to or is non-responsive to induction chemotherapy, the method comprising:
determining the expression level of FOXC1 in a sample obtained from the patient, wherein an elevated level of FOXC1 expression indicates that the patient is unlikely to respond to or is non-responsive to induction chemotherapy.

2. The method of claim 1, wherein the sample is a tissue sample or a body fluid sample.

3. The method of claim 1, wherein the sample is a bone marrow sample.

4. The method of claim 1, wherein the sample is a blood sample.

5. The method of claim 1, wherein the AML patient has been subjected to induction chemotherapy.

6. The method of claim 1, wherein the AML patient has been diagnosed with AML but has not been subjected to induction therapy.

7. A method of treating an AML patient who is unlikely to respond to or who is non-responsive to induction chemotherapy, the method comprising:
detecting the expression level of FOXC1 from a sample obtained from an AML patient; and
administering a therapeutically effective amount of one or more alternative therapies that is not chemotherapy to the patient who has an elevated level of FOXC1 expression.

8. The method of claim 7, wherein the alternative therapy includes stem cell transplantation, radiotherapy, or a targeted therapy.

9. The method of claim 7, wherein the sample is a tissue sample or a body fluid sample.

10. The method of claim 7, wherein the sample is a bone marrow sample.

11. The method of claim 7, wherein the sample is a blood sample.

12. The method of claim 7, wherein the AML patient has been subjected to induction chemotherapy.

13. The method of claim 7, wherein the AML patient has been diagnosed with AML but has not been subjected to induction chemotherapy.

14. The method of claim 7, wherein the alternative therapy is stem cell transplantation, and the stem cells are autologous stem cells.

15. The method of claim 7, wherein the alternative therapy is stem cell transplantation, and the stem cells are allogeneic stem cells.

16. A method of treating an AML patient who is likely to respond to or who is responsive to induction chemotherapy, the method comprising:
detecting the expression level of FOXC1 from a sample obtained from an AML patient, and
administering a therapeutically effective amount of chemotherapy to the AML patient who does not have an elevated expression level of FOXC1.

17. The method of claim 16, wherein the sample is a tissue sample or a body fluid sample.

18. The method of claim 16, wherein the sample is a bone marrow sample.

19. The method of claim 16, wherein the sample is a blood sample.
